(19) United States
(12) Patent Application Publication
(10) Pub. No.: US 2026/0273016 A1
(43) Pub. Date: Sep. 17, 2026

(54) STABLE LIQUID BORTEZOMIB FORMULATIONS

(71) Applicant: Riconpharma LLC, Denville, NJ (US)

(72) Inventors: Mukteeshwar Gande, Monroe, NJ (US); Praveen Reddy Billa, Flanders, NJ (US); SVB Janardhan Garikipati, Hyderabad (IN); Vinay Sudhindra Raichur, Hyderabad (IN); Vijaya Kumar Thota, Hyderabad (IN)

(73) Assignee: Riconpharma LLC, Denville, NJ (US)

(21) Appl. No.: 19/682,935

(22) Filed: May 20, 2026

Related U.S. Application Data
(63) Continuation-in-part of application No. 19/341,758, filed on Sep. 26, 2025, now abandoned.
(60) Provisional application No. 63/699,663, filed on Sep. 26, 2024.

(51) Int. Cl. A61K 38/05; A61K 9/08; A61K 47/02; A61K 47/26

(52) U.S. Cl. CPC A61K 38/05; A61K 9/08; A61K 47/02; A61K 47/26

(57) ABSTRACT

Described herein are liquid pharmaceutical compositions comprising bortezomib, a polyol comprising mannitol, and an aqueous carrier, wherein the compositions are suitable for intravenous or subcutaneous administration. In certain embodiments, the compositions are substantially free of organic solvents and comprise controlled levels of impurities, including not more than 1.0% of each of Impurity E, Impurity L, Impurity J, Impurity F, Impurity I, and Impurity U, and not more than 3.0% total impurities. The compositions are prepared under controlled process conditions, including control of temperature, dissolved oxygen, and headspace oxygen, to reduce impurity formation and improve stability during storage. In certain embodiments, the compositions exhibit extended stability under refrigerated conditions. The disclosure further relates to methods of preparing such compositions and to dosage configurations aligned with clinically administered doses to improve dose matching and reduce residual drug following administration.

### CROSS-REFERENCE TO RELATED APPLICATIONS

This application is a continuation-in-part of U.S. application Ser. No. 19/341,758, filed Sep. 26, 2025, which claims priority from U.S. Provisional Application No. 63/699,663, filed Sep. 26, 2024, the disclosures of which are incorporated by reference herein in their entireties.

### FIELD OF THE DISCLOSURE

The present disclosure relates to liquid pharmaceutical formulations of bortezomib and to processes for preparing such formulations. More particularly, this disclosure relates to stable, pharmaceutically acceptable liquid bortezomib compositions prepared in an aqueous medium and substantially free of organic solvents using controlled process conditions involving mechanical mixing or high shear homogenization under controlled temperature. This disclosure further relates to dosage configurations aligned with clinically administered doses to improve dose matching and reduce residual drug during administration.

### BACKGROUND

Bortezomib is a proteasome inhibitor indicated for the treatment of adult patients with multiple myeloma and mantle cell lymphoma. Currently approved formulations of bortezomib are predominantly lyophilized powder presentations, wherein the reference listed drug (RLD), VELCADE®, is available as a 3.5 mg/vial product. Additional presentations, including other approved strengths, are also available. These lyophilized formulations require reconstitution with a suitable diluent, such as 0.9% sodium chloride solution, prior to administration by subcutaneous or intravenous routes.

The requirement for reconstitution introduces several practical limitations in clinical settings, including increased preparation steps, potential for dosing variability, and risk of handling errors. Further, such fixed-dose vial presentations, when used in conjunction with body surface area (BSA)-based dosing regimens, may result in incomplete utilization of the reconstituted drug product, leading to residual unused drug and associated inefficiencies in drug utilization. Accordingly, there has been continued interest in developing liquid, ready-to-use bortezomib formulations that can simplify administration and improve dosing efficiency.

Bortezomib is known to be susceptible to degradation under certain environmental and processing conditions. Such degradation pathways may be influenced not only by formulation composition but also by process conditions employed during preparation. As a result, controlling degradation and maintaining acceptable impurity profiles in liquid bortezomib formulations remains a significant challenge, particularly for formulations intended for extended storage.

Other liquid bortezomib formulations include combinations of solvents, stabilizers, antioxidants, or buffering systems to address stability concerns. However, these formulations generally rely on complex excipient systems and do not adequately address the role of process conditions.

In particular, existing approaches do not sufficiently address the combined influence of manufacturing conditions, including thermal exposure, mixing intensity and dissolved or headspace oxygen levels, on degradation pathways associated with liquid bortezomib formulations. As a result, maintaining low impurity levels over extended storage periods remains a challenge.

Accordingly, there remains a need for liquid bortezomib formulations that can be prepared in an aqueous system substantially free of organic solvents, while maintaining acceptable impurity profiles and stability during storage.

Additionally, conventional fixed-dose presentations may not be optimally aligned with clinically administered, body surface area-based dosing regimens, resulting in incomplete utilization of the drug product and associated wastage. Accordingly, there remains a need for stable liquid bortezomib formulations that combine improved process-controlled stability with dosage configurations that enhance dose matching and reduce residual drug during administration.

Certain embodiments of the present invention are designed to address these deficiencies and provide other clinically significant benefits.

### SUMMARY

The present inventors have discovered that elevated temperatures, oxygen, and light can lead to the formation of degradation impurities and a reduction in assay over time.

In an effort to address limitations associated with lyophilized formulations, various liquid bortezomib formulations have been developed. Although certain ready-to-use liquid formulations comprising bortezomib and pharmaceutically acceptable excipients may be known, such formulations often employ organic solvents or co-solvent systems, including alcohols (e.g., tert-butyl alcohol), and may involve multi-step processing such as solvent dissolution at elevated temperatures, solvent removal or evaporation, or lyophilization. The presence of such solvent systems can increase process complexity and may contribute to degradation pathways or require additional controls to maintain acceptable impurity profiles in the final product.

Some embodiments of the present invention relate to liquid pharmaceutical compositions of bortezomib and to processes for preparing such compositions. In certain embodiments, the present invention provides stable, pharmaceutically acceptable liquid bortezomib compositions prepared in an aqueous medium under controlled process conditions.

In certain embodiments, the compositions are substantially free of organic solvents and are prepared without reliance on organic solvent-based processing steps. In some embodiments, the compositions comprise bortezomib, a polyol such as mannitol, and optionally a tonicity adjusting agent, in an aqueous medium.

In certain embodiments, dissolution of bortezomib in the aqueous medium is achieved using mechanical energy, including high shear homogenization, without reliance on elevated temperature or organic solvents. In some embodiments, high shear homogenization is performed at shear rates corresponding to about 15 Hz to about 40 Hz under controlled temperature conditions. In certain embodiments, such mechanical processing facilitates rapid dispersion and dissolution of bortezomib despite its low aqueous solubility and poor wettability and thus leads to formation of a visually clear solution. In some embodiments, the process comprises preparing an aqueous solution comprising a polyol, controlling dissolved oxygen levels in the solution, adding bortezomib under controlled temperature conditions, and applying mechanical mixing or high shear homogenization to obtain a clear solution, followed by filling the composition into a container under controlled atmospheric conditions.

The present inventors discovered that dissolution using elevated temperature may lead to increased formation of degradation impurities, whereas dissolution using mechanical energy, including high shear homogenization, reduces such impurity formation.

In certain embodiments, the liquid bortezomib compositions prepared according to the processes described herein exhibit reduced formation of degradation impurities and improved stability during storage. In some embodiments, the compositions maintain acceptable assay and impurity levels over extended storage periods under refrigerated conditions.

In certain embodiments, compositions of the present invention maintain controlled impurity profiles, including low levels of degradation products such as Impurity E, Impurity F, Impurity L and related species, during storage. In some embodiments, total impurities are maintained within acceptable limits over the storage period.

In other embodiments, the present invention provides liquid bortezomib compositions in dosage configurations aligned with clinically administered doses. In some embodiments, the compositions are provided at a concentration of about 2.5 mg/mL in a fill volume corresponding to a total drug content of about 3 mg.

In further embodiments, such dosage configurations improve dose matching for body surface area-based dosing regimens and reduce residual drug remaining after administration. In certain embodiments, the amount of unused drug remaining after preparation of a required dose is reduced relative to higher-strength presentations.

In some embodiments, the present invention provides a liquid bortezomib composition prepared in accordance with any one of the methods described herein.

In other embodiments, the present invention provides liquid bortezomib compositions and processes that combine process-controlled stability with improved dosage alignment, thereby enhancing drug utilization efficiency while maintaining acceptable impurity profiles and stability during storage.

### DETAILED DESCRIPTION

In the context of this invention, “bortezomib” refers to pharmaceutically acceptable salts, solvates, hydrates, and anhydrous forms of [(1R)-3-methyl-1-[[(2S)-1-oxo-3-phenyl-2-[(pyrazinylcarbonyl)amino]propyl]amino]butyl]boronic acid. The structure of bortezomib is provided below.

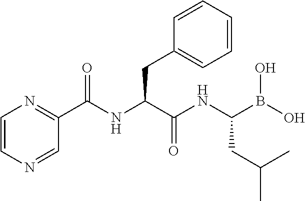 [SMILES: CC(C)C[C@H](NC(=O)[C@H](Cc1ccccc1)NC(=O)c1cnccn1)B(O)O]

As used herein, “liquid bortezomib” or “ready-to-use bortezomib” formulations refer to formulations comprising bortezomib in dissolved or solubilized form, suitable for administration via subcutaneous route or, optionally, dilution with a pharmaceutically acceptable diluent, preferably 0.9% sodium chloride solution, for intravenous administration.

In certain embodiments, the concentration of bortezomib is about 2.5 mg/mL for subcutaneous administration and about 1 mg/mL for intravenous administration.

In certain embodiments, bortezomib forms a boronic acid ester in the presence of a polyol, such as mannitol, contributing to improved solubility and stability. The compositions may comprise bortezomib and mannitol in a system wherein the ester and its hydrolysis product exist in equilibrium.

In certain embodiments, bortezomib forms a reversible boronic acid ester with the polyol, such as mannitol, wherein the ester and the corresponding free boronic acid exist in equilibrium in the aqueous medium. Such equilibrium may be present in known formulations comprising bortezomib and polyols. However, it has been found that the presence of such equilibrium alone is not sufficient to achieve the stability characteristics described herein. In particular, degradation pathways are governed by process conditions, including temperature and oxygen exposure during preparation and filling and thereby contribute to the improved stability observed in the compositions described herein.

In certain embodiments, the concentration of bortezomib ranges from about 1 mg/mL to about 2.5 mg/mL, the concentration of mannitol ranges from about 5 mg/mL to about 50 mg/mL, and the concentration of sodium chloride ranges from about 5 mg/mL to about 18 mg/mL.

As used herein, “dissolved oxygen (DO)” refers to oxygen present in the liquid phase of the composition, expressed in parts per million (ppm).

“Headspace oxygen (HSO)” refers to oxygen present in the gaseous space above the liquid composition within a container, expressed as volume percentage.

“Residual drug” refers to the amount of drug remaining unused after withdrawal of a required dose from a container.

As used herein, ‘substantially free of organic solvents’ means that the composition contains no intentionally added organic solvent and, if present, residual solvent levels are below pharmaceutically acceptable limits.

As used herein, “controlled process conditions” refers to simultaneous control of process parameters during preparation and/or filling of the composition using mechanical mixing, or high shear homogenization, including:
(i) temperature not exceeding about 30° C.;
(ii) dissolved oxygen at not more than about 5 ppm;
(iii) headspace oxygen at not more than about 5%; and
(iv) high shear homogenization performed at shear rates corresponding to about 15 Hz to about 40 Hz, such that degradation associated with thermal and oxidative pathways is minimized.

As used herein, “refrigerated storage conditions” refers to storage at a temperature of about 2° C. to about 8° C.

As used herein, “mechanical mixing” or “high shear homogenization” refers to application of mechanical energy to the composition sufficient to promote dispersion and dissolution of bortezomib in the aqueous medium, including through shear forces that reduce particle size and improve wettability. In certain embodiments, such mixing is performed at shear rates corresponding to about 15 Hz to about 40 Hz or equivalent mixing intensity.

As used herein, “stable” or “stability” refers to the ability of the composition to maintain:
(i) assay within about 90% to about 110% of the labelled amount; and
(ii) impurity levels within specified limits, including total impurities not exceeding predefined thresholds, over a specified storage period under defined storage conditions.

In certain embodiments, the present invention provides a process for preparing a liquid bortezomib composition comprising dissolving bortezomib in an aqueous medium containing a polyol, such as mannitol. In certain embodiments, dissolution may be facilitated at temperatures in the range of about 25° C. to about 90° C. depending on polymorphic form. In certain embodiments, dissolution is facilitated using mechanical mixing or high shear homogenization performed at shear rates corresponding to about 15 Hz to about 40 Hz. It has been found that limiting temperature exposure during preparation contributes to an improved impurity profile and stability.

In certain embodiments, the compositions are prepared in an aqueous system substantially free of organic solvents. However, it has been observed that the absence of organic solvents alone is not sufficient to achieve the desired impurity profile, and that control of process parameters, including temperature and oxygen levels, is required to obtain compositions having optimal stability.

In certain embodiments, dissolution of bortezomib is achieved using mechanical energy rather than thermal means. In some embodiments, high shear homogenization is employed to facilitate dispersion and dissolution of bortezomib in the aqueous medium.

Bortezomib exhibits low aqueous solubility and poor wettability, which can limit dissolution under conventional mixing conditions. It has been found that application of mechanical energy, including high shear homogenization, promotes particle size reduction and improved dispersion, resulting in enhanced dissolution without the need for elevated temperature or organic solvents.

In certain embodiments, the homogenization is carried out at a shear rate corresponding to about 15 Hz to about 40 Hz.

Bortezomib drug substance may be present in different polymorphic forms, including Form 1 and Form 2, and dissolution conditions may vary accordingly.

In certain embodiments, it has been found through systematic evaluation that process conditions employed during preparation of liquid bortezomib compositions significantly influence impurity formation and long-term stability. In particular, it was observed that exposure to elevated temperatures and uncontrolled oxygen levels during processing contributes to the formation of degradation impurities associated with thermal and oxidative pathways.

It has been observed that dissolution of bortezomib using elevated temperatures, while improving solubility, may lead to increased formation of degradation impurities, including Impurity L, Impurity J, and Impurity F, both initially and during storage.

In contrast, dissolution using mechanical energy, including high shear homogenization, performed at shear rates corresponding to about 15 Hz to about 40 Hz, enables formation of a clear solution without exposure to elevated temperatures, thereby reducing temperature-induced degradation pathways.

In certain embodiments, the process employs mechanical energy in combination with controlled oxygen conditions to achieve reduced impurity formation and improved long-term stability.

In certain embodiments, the process is carried out under controlled conditions selected to reduce impurity formation. In some embodiments, the composition is prepared under conditions wherein exposure to elevated temperatures is reduced or minimized during one or more stages of preparation.

In certain embodiments, the process is conducted at a temperature not exceeding about 40° C., preferably not exceeding about 30° C.

In certain embodiments, the absence of organic solvents may reduce solvent-related degradation pathways; however, it has been found that such absence alone is insufficient to achieve the desired impurity profile without simultaneous control of process parameters (e.g., temperature, dissolved oxygen, and headspace oxygen).

In certain embodiments, dissolved oxygen present in the aqueous medium is reduced prior to and/or during preparation. In some embodiments, dissolved oxygen levels are maintained at not more than about 10 ppm, preferably not more than about 5 ppm, and more preferably not more than about 2 ppm.

In certain embodiments, headspace oxygen within the container is controlled during and/or after filling. In some embodiments, headspace oxygen is maintained at not more than about 5%, preferably not more than about 2%, and more preferably not more than about 1%.

The present inventors further observed that achieving the desired impurity profile required simultaneous control of temperature, dissolved oxygen, and headspace oxygen, and that control of any one parameter alone was not sufficient. This demonstrates that stability is governed by a combination of process parameters rather than any single variable.

In certain embodiments, the process comprises: (i) providing an aqueous solution comprising a polyol and optionally a tonicity adjusting agent; (ii) reducing dissolved oxygen in the solution; (iii) adding bortezomib under controlled temperature conditions; (iv) applying mechanical mixing, including high shear homogenization, at shear rates corresponding to about 15 Hz to about 40 Hz, to obtain a clear solution without reliance on elevated temperature; (iv) applying mechanical mixing, including high shear homogenization, and (v) filling the composition into a container under controlled atmospheric conditions.

In certain embodiments, compositions prepared under the process conditions described herein exhibit reduced formation of degradation impurities and improved stability during storage.

In certain embodiments, total impurities are maintained below about 2.5%, preferably below about 2.0%, preferably below about 1.5% and more preferably below about 1.0% during storage at refrigerated temperature.

In certain embodiments, impurities include degradation products arising from thermal, oxidative, or process-related pathways, including but not limited to Impurity E, Impurity F, Impurity L and Impurity J.

The chemical structures of representative impurities are provided below.

### Impurity E:

Chemical name: (S)—N-(1-amino-1-oxo-3-phenylpropan-2-yl)pyrazine-2-carboxamide

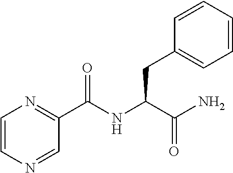 [SMILES: NC(=O)[C@H](Cc1ccccc1)NC(=O)c1cnccn1]

### Impurity F:

Chemical name: N—((S)-1-((R)-1-hydroxy-3-methylbutylamino)-1-oxo-3-phenylpropan-2-yl)pyrazine-2-carboxamide

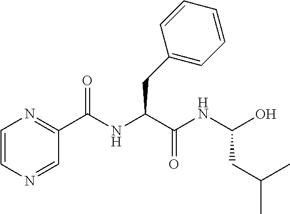 [SMILES: CC(C)C[C@@H](O)NC(=O)[C@H](Cc1ccccc1)NC(=O)c1cnccn1]

### Impurity I:

Chemical name: N—((R)-1-((R)-1-hydroperoxy-3-methylbutyl amino)-1-oxo-3-phenylpropan-2-yl)pyrazine-2-carboxamide

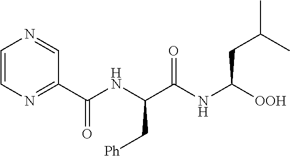

### Impurity J:

Chemical name: N—((S)-1-((S)-1-hydroxy-3-methylbutylamino)-1-oxo-3-phenylpropan-2-yl)pyrazine-2-carboxamide

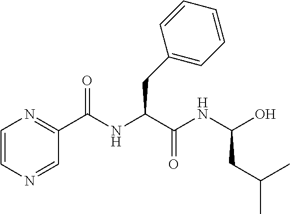 [SMILES: CC(C)C[C@H](O)NC(=O)[C@H](Cc1ccccc1)NC(=O)c1cnccn1]

### Impurity L:

Chemical name: (S)-3-phenyl-2-(pyrazine-2-carboxamido) propanoic acid

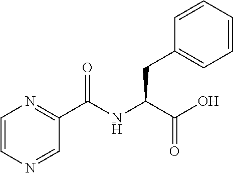 [SMILES: O=C(N[C@@H](Cc1ccccc1)C(=O)O)c1cnccn1]

### Impurity U:

Chemical name: (R)—N-(1-(3-methylbutanamido)-1-oxo-3-phenylpropan-2-yl) pyrazine-2-carboxamide

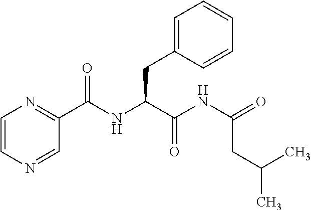 [SMILES: CC(C)CC(=O)NC(=O)[C@H](Cc1ccccc1)NC(=O)c1cnccn1]

In certain embodiments, the compositions described herein exhibit low initial levels of degradation impurities, including Impurity E, Impurity L, Impurity J and Impurity F and maintain such levels during storage under refrigerated conditions.

In certain embodiments, the composition exhibits controlled impurity levels during storage. In some embodiments, the impurity profile remains substantially comparable to initial levels over the storage period, thereby indicating improved stability of the formulation.

Without being bound by theory, it is believed that exposure of bortezomib to elevated temperatures during preparation may promote formation of thermal degradation products, while the presence of oxygen may contribute to oxidative degradation pathways. In certain embodiments, reducing thermal exposure, applying controlled mechanical mixing or high shear homogenization, and controlling dissolved oxygen and/or headspace oxygen during preparation and filling suppresses such degradation pathways, thereby contributing to improved impurity profile and stability of the composition.

In certain embodiments, liquid bortezomib compositions prepared under the process conditions described herein remain stable under refrigerated storage conditions for extended periods, including at least about 12 months, at least about 18 months, or at least about 24 months at a temperature of about 2° C. to about 8° C. Such stability profiles are consistent with experimental data obtained from representative compositions prepared under the process conditions described herein.

In certain embodiments, total impurities in the composition of present invention are maintained at not more than about 2.5%, preferably not more than about 2.0%, preferably not more than about 1.5% and more preferably not more than 1.0% after storage under refrigerated conditions. In some embodiments, other impurities, such as, Impurity E, Impurity J, or Impurity F are not more than about 0.3 wt. % and Impurity L is not more than about 0.5 wt. % after storage under refrigerated conditions for extended periods.

In certain embodiments, individual impurities, including those arising from thermal and/or oxidative degradation pathways, are maintained within acceptable limits during storage.

In certain embodiments, liquid bortezomib compositions prepared under controlled process conditions, including reduced temperature exposure, controlled mechanical mixing or high shear homogenization, and controlled dissolved oxygen and/or headspace oxygen levels, and in an aqueous system substantially free of organic solvents, exhibit improved stability over extended storage durations. In some embodiments, the composition maintains acceptable assay and impurity levels for at least about 12 months, and in certain embodiments at least about 24 months under refrigerated conditions.

In certain embodiments, liquid bortezomib compositions prepared under process conditions involving elevated temperature exposure and/or organic solvent-based processing, including solvent removal steps, exhibit acceptable stability over shorter storage durations, for example up to about 3 months.

In certain embodiments, liquid bortezomib compositions may be prepared using different process approaches. However, it has been found that compositions prepared under controlled process conditions, including controlled mechanical mixing or high shear homogenization conditions, as described herein exhibit improved impurity profile and extended stability compared to compositions prepared under less controlled conditions.

In certain embodiments, organic solvent-based processing may also be employed, and the stability and impurity profile of the resulting formulation may vary depending on the process conditions employed.

In certain embodiments, the organic solvent-based processing comprises dissolving bortezomib in a solvent system comprising an organic solvent, followed by solvent removal and subsequent dilution to obtain the final composition.

In certain embodiments, compositions of the present invention are provided in a container selected from a vial, pre-filled syringe, or cartridge. In some embodiments, the container comprises a single-dose presentation suitable for withdrawal of a therapeutically effective amount of the composition. In certain embodiments, the container is configured to maintain controlled headspace oxygen levels and to facilitate withdrawal of the composition for subcutaneous or intravenous administration. As used herein, “container suitable for subcutaneous or intravenous administration” refers to a container that enables withdrawal of a therapeutically effective dose of the composition for direct administration or dilution, including but not limited to vials, pre-filled syringes, or cartridges.

In certain embodiments, the liquid bortezomib compositions described herein are provided in dosage configurations aligned with clinically administered doses. In certain embodiments, such dosage configurations are enabled by the improved stability of the compositions. In certain embodiments, the improved stability enables provision of liquid bortezomib compositions in dosage configurations that are aligned with clinically administered doses, thereby facilitating improved dose utilization and reduced residual drug.

Bortezomib is typically administered based on body surface area (BSA), and fixed vial strengths may not correspond precisely to required patient doses, which may result in residual unused drug following administration.

In certain embodiments, the present invention provides dosage configurations selected based on evaluation of drug utilization across a clinically relevant range of BSA values.

In certain embodiments, the composition is provided at a concentration of about 2.5 mg/mL in a fill volume corresponding to a total drug content of about 3 mg.

In certain embodiments, such dosage configurations improve dose alignment and reduce residual drug remaining after administration.

In certain embodiments, the dosage configuration differs from conventional 3.5 mg presentations and provides improved alignment with clinically administered doses.

In certain embodiments, the residual drug remaining after administration is reduced by at least about 10%, preferably at least about 15%, and more preferably at least about 20% compared to higher-strength presentations across a body surface area range of about 1.4 m² to about 2.2 m². In certain embodiments, the reduction in residual drug may range from about 30% to about 80% depending on patient body surface area. In certain embodiments, such reduction in residual drug is achieved without compromising the stability and impurity profile of the composition. In certain embodiments, the reduction in residual drug is determined by comparing the amount of unused drug remaining after preparation of a required dose from different vial strengths.

In certain embodiments, the dosage configurations described herein improve utilization efficiency while maintaining stability and performance characteristics of the composition.

In some embodiments, the present invention provides liquid pharmaceutical compositions comprising: from about 1 mg/mL to about 5 mg/mL bortezomib; from about 15 mg/mL to about 40 mg/mL mannitol; from about 5 mg/mL to about 18 mg/mL sodium chloride; and an aqueous carrier; wherein the composition is substantially free of organic solvents; and wherein the composition is prepared by a process comprising the following steps:
(a) preparing a solution comprising the mannitol, the aqueous carrier, and the sodium chloride;
(b) purging the solution from step (a) with an inert gas;
(c) adding the bortezomib to the solution of step (b) at a temperature not exceeding about 30° C.;
(d) mixing the product of step (c) at a shear rate of from about 15 Hz to about 40 Hz until a clear solution is obtained; and
(e) filling the product of step (d) in a vial under an inert gas atmosphere; and wherein after storage for at least about 12 months at about 2° C. to about 8° C., the composition comprises: not more than about 0.3 wt. % of Impurity E, Impurity J, or Impurity F, individually; not more than about 0.5 wt. % of Impurity L; and not more than about 1.0 wt. % totally impurities.

### EXAMPLES

### Example 1: Preparation of Ready to Use Aqueous Solution of Bortezomib (Form 1) at 90° C.

Approximately 2.5 g of mannitol was weighed and added to 90.0 g of water with continuous stirring. To the solution, 0.9 g of sodium chloride was added and stirred continuously to obtain a clear solution. The solution was purged with nitrogen to maintain the dissolved oxygen below 2 ppm. The solution was heated to a temperature of 90° C. To the heated solution, bortezomib (Form 1) API, 0.25 g was added under continuous stirring to form a clear solution. The solution was cooled down to room temperature and final volume of the solution was made up to 100 g. All the operations were performed under sodium vapor lamp to minimize photo degradation. The solution was filtered through a 0.2μ filter. 1.0 mL of solution was filled into each vial, stoppered with a rubber stopper and sealed with flip off seals.

TABLE 1

| Condition | Specification | Initial |
|---|---|---|
| Assay | 90%-110% | 100.7 |
| Impurity-E | NMT 1.0% | 0.02 |
| Impurity-L | NMT 1.0% | 0.31 |
| Impurity-J | NMT 1.0% | 0.04 |
| Impurity-F | NMT 1.0% | 0.05 |
| Impurity-I | NMT 1.0% | ND |
| Impurity-U | NMT 1.0% | ND |
| Highest Unknown | NMT 0.5% | 0.01 |
| Total impurities | NMT 3.0% | 0.47 |

### Example 2: Preparation of Ready to Use Aqueous Solution of Bortezomib (Form 1) at 80° C.

Approximately 10.0 g of mannitol was weighed and added to 360.0 g of water with continuous stirring. To the solution 3.6 g of sodium chloride was added and stirred continuously to obtain a clear solution. The solution was purged with nitrogen to maintain the dissolved oxygen below 2 ppm. The solution was heated to a temperature of 80° C. To the heated solution, bortezomib (Form 1) API 1.00 g was added under continuous stirring to form a clear solution. The solution was cooled down to room temperature and final volume of the solution was made up to 400 g. All the operations were performed under sodium vapor lamp to minimize photo degradation. The solution was filtered through a 0.2μ filter. 1.4 mL of solution was filled into each vial, stoppered with a rubber stopper and sealed with flip off seals.

TABLE 2

| Condition | Specification | Initial | 2-8° C. (2 M) |
|---|---|---|---|
| Assay | 90%-110% | 100.8 | 102.1 |
| Impurity-E | NMT 1.0% | 0.02 | 0.07 |
| Impurity-L | NMT 1.0% | 0.17 | 0.38 |
| Impurity-J | NMT 1.0% | 0.03 | 0.08 |
| Impurity-F | NMT 1.0% | 0.05 | 0.08 |
| Impurity-I | NMT 1.0% | ND | 0.08 |
| Impurity-U | NMT 1.0% | ND | 0.01 |
| Highest Unknown | NMT 0.5% | 0.01 | 0.01 |
| Total impurities | NMT 3.0% | 0.31 | 0.78 |

### Example 3: Preparation of Ready to Use Aqueous Solution of Bortezomib (Form 1) at 60° C.

Approximately 5.00 g of mannitol was weighed and added to 180.0 g of water with continuous stirring. To the solution, 1.80 g of sodium chloride was added and stirred continuously to obtain a clear solution. The solution was purged with nitrogen to maintain the dissolved oxygen below 2 ppm. The solution was heated to a temperature of 60° C. To the heated solution, bortezomib (Form 1) API 0.5 g was added under continuous stirring to form a clear solution. The solution was cooled down to room temperature and final volume of the solution was made up to 200 g. All the operations were performed under sodium vapor lamp to minimize photo degradation. The solution was filtered through a 0.2μ filter. 1.4 mL of solution was filled into each vial, stoppered with a rubber stopper and sealed with flip off seals.

TABLE 3

| Condition | Specification | Initial |
|---|---|---|
| Assay | 90%-110% | 97.8 |
| Impurity-E | NMT 1.0% | 0.02 |
| Impurity-L | NMT 1.0% | 0.23 |
| Impurity-J | NMT 1.0% | 0.06 |
| Impurity-F | NMT 1.0% | 0.08 |
| Impurity-I | NMT 1.0% | ND |
| Impurity-U | NMT 1.0% | ND |
| Highest Unknown | NMT 0.5% | 0.01 |
| Total impurities | NMT 3.0% | 0.45 |

### Example 4: Preparation of Ready to Use Aqueous Solution of Bortezomib (Form 2) at 50° C.

Approximately 2.50 g of mannitol was weighed and added to 90.0 g of water with continuous stirring. To the solution, 0.9 g of sodium chloride was added and stirred continuously to obtain a clear solution. The solution was purged with nitrogen to maintain the dissolved oxygen below 2 ppm. The solution was heated to a temperature of 50° C. To the heated solution, bortezomib (Form 2) API 0.25 g was added under continuous stirring to form a clear solution. The solution was cooled down to room temperature and final volume of the solution was made up to 100 g. All the operations were performed under sodium vapor lamp to minimize photo degradation. The solution was filtered through a 0.2μ filter. 1.4 mL of solution was filled into each vial, stoppered with a rubber stopper and sealed with flip off seals.

TABLE 4

| Condition | Specification | Initial | 2° C. to 8° C. (2 weeks) |
|---|---|---|---|
| Assay | 90%-110% | 100.9 | 101.4 |
| Impurity-E | NMT 1.0% | 0.07 | 0.07 |
| Impurity-L | NMT 1.0% | 0.06 | 0.11 |
| Impurity-J | NMT 1.0% | 0.06 | 0.07 |
| Impurity-F | NMT 1.0% | 0.07 | 0.07 |
| Impurity-I | NMT 1.0% | ND | 0.03 |
| Impurity-U | NMT 1.0% | ND | ND |
| Highest Unknown | NMT 0.5% | 0.01 | 0.01 |
| Total impurities | NMT 3.0% | 0.35 | 0.39 |

### Example 5: Preparation of Ready to Use Aqueous Solution of Bortezomib (Form 2) at 40° C.

Approximately 2.50 g of mannitol was weighed and added to 90.0 g of water with continuous stirring. To the solution, 0.9 g of sodium chloride was added and stirred continuously to obtain a clear solution. The solution was purged with nitrogen to maintain the dissolved oxygen below 2 ppm. The solution was heated to a temperature of 40° C. To the heated solution, bortezomib (Form 2) API 0.25 g was added under continuous stirring to form a clear solution. The solution was cooled down to room temperature and final volume of the solution was made up to 100 g. All the operations were performed under sodium vapor lamp to minimize photo degradation. The solution was filtered through a 0.2μ filter. 1.4 mL of solution was filled into each vial, stoppered with a rubber stopper and sealed with flip off seals.

TABLE 5

| Condition | Specification | Initial | 2° C. to 8° C. (2 weeks) |
|---|---|---|---|
| Assay | 90%-110% | 99.7 | 101.3 |
| Impurity-E | NMT 1.0% | 0.03 | 0.07 |
| Impurity-L | NMT 1.0% | 0.03 | 0.14 |
| Impurity-J | NMT 1.0% | 0.06 | 0.12 |
| Impurity-F | NMT 1.0% | 0.08 | 0.12 |
| Impurity-I | NMT 1.0% | 0.02 | 0.02 |
| Impurity-U | NMT 1.0% | ND | 0.01 |
| Highest Unknown | NMT 0.5% | 0.01 | 0.01 |
| Total impurities | NMT 3.0% | 0.23 | 0.53 |

### Example 6: Preparation of Ready to Use Aqueous Solution of Bortezomib (Form 2) at RT

Approximately 2.5 g of mannitol was weighed and added to 90.0 g of water with continuous stirring. To the solution, 0.9 g of sodium chloride was added and stirred continuously to obtain a clear solution. The solution was purged with nitrogen to maintain the dissolved oxygen below 2 ppm. To the solution, bortezomib (Form 2) API 0.25 g was added under continuous stirring to form a clear solution. The solution was cooled down to room temperature and final volume of the solution was made up to 100 g. All the operations were performed under sodium vapor lamp to minimize photo degradation. The solution was filtered through a 0.2μ filter. 1.4 mL of solution was filled into each vial, stoppered with a rubber stopper and sealed with flip off seals.

TABLE 6

| Condition | Specification | Initial |
|---|---|---|
| Assay | 90%-110% | 98.3 |
| Impurity-E | NMT 1.0% | 0.03 |
| Impurity-L | NMT 1.0% | 0.02 |
| Impurity-J | NMT 1.0% | 0.05 |
| Impurity-F | NMT 1.0% | 0.09 |
| Impurity-I | NMT 1.0% | ND |
| Impurity-U | NMT 1.0% | ND |
| Highest Unknown | NMT 0.5% | 0.01 |
| Total impurities | NMT 3.0% | 0.21 |

### Example 7: Preparation of Ready to Use Aqueous Solution of Bortezomib (Form 1) at 80° C. Without Sodium Chloride

Approximately 2.5 g of mannitol was weighed and added to 90.0 g of water with continuous stirring. The solution was heated to a temperature of 80° C. To the heated solution, bortezomib (Form 1) API 0.25 g was added under continuous stirring to form a clear solution. The solution was cooled down to room temperature and final volume of the solution was made up to 100 g. The solution was filtered through a 0.2μ filter. 1.0 mL of solution was filled into each vial, stoppered with a rubber stopper and sealed with flip off seals.

TABLE 7

| Condition | Specification | Initial | 2° C. to 8° C. (3M) |
|---|---|---|---|
| Assay | 90%-110% | 95.8 | 97.3 |
| Impurity-E | NMT 1.0% | 0.02 | 0.02 |
| Impurity-L | NMT 1.0% | 0.19 | 0.26 |
| Impurity-J | NMT 1.0% | 0.06 | 0.09 |
| Impurity-F | NMT 1.0% | 0.06 | 0.07 |
| Impurity-I | NMT 1.0% | ND | ND |
| Impurity-U | NMT 1.0% | ND | ND |
| Highest Unknown | NMT 0.5% | ND | ND |
| Total impurities | NMT 3.0% | 0.38 | 0.49 |

### Example 8: Preparation of Ready to Use Aqueous Solution of Bortezomib (Form 1) with Vacuum Evaporation Process with 0.9% Sodium Chloride

### Preparation of Concentrate:

Step 1: Approximately 330 g of water was taken and to it added 24.75 g of mannitol under continuous stirring to form a clear solution. The solution obtained was then purged with nitrogen to obtain a dissolved oxygen (DO) level below 2%.

Step 2: Approximately 171.6 g of tert-butyl alcohol was taken and 2.556 g of bortezomib was added under stirring at a temperature of 50° C. to obtain a clear solution.

Step 3: The products of Step 1 and Step 2 were mixed together and charged for vacuum evaporation in Buchi Rotavap at vacuum of 300 mbar and gradually reduced to 150 mbar and then to 60 mbar.

### Preparation of Final Formulation:

Step 4: 207.65 g of concentrate was taken and made up to 1000 g. The batch was divided into two parts and 0.9% sodium chloride was added to one part. The solution was then filtered through a 0.2μ filter.

TABLE 8

| Condition | Specification | Initial | 2° C. to 8° C. (3M) |
|---|---|---|---|
| Assay | 90%-110% | 97.9 | 97.5 |
| Impurity-E | NMT 1.0% | 0.01 | 0.01 |
| Impurity-L | NMT 1.0% | 0.03 | 0.08 |
| Impurity-J | NMT 1.0% | 0.06 | 0.11 |
| Impurity-F | NMT 1.0% | 0.10 | 0.12 |
| Impurity-I | NMT 1.0% | ND | ND |
| Impurity-U | NMT 1.0% | ND | ND |
| Highest Unknown | NMT 0.5% | 0.01 | 0.01 |
| Total impurities | NMT 3.0% | 0.20 | 0.32 |

### Example 9: Preparation of Ready to Use Aqueous Solution of Bortezomib (Form 1) with Vacuum Evaporation Process without Sodium Chloride

### Preparation of Concentrate:

Step 1: Approximately 57.798 g of water was taken and to it added 5.032 g of mannitol under continuous stirring to form a clear solution. The solution obtained was then purged with nitrogen to obtain DO level below 2%.

Step 2: Approximately 32.002 g of tert-butyl alcohol was taken and to it added 0.501 g of bortezomib under stirring at a temperature of 50° C. to obtain a clear solution.

Step 3: The products of Step 1 and Step 2 were mixed together and charged for vacuum evaporation in Buchi Rotavap at vacuum of 300 mbar and gradually reduced to 150 mbar and then to 60 mbar.

### Preparation of Final Formulation:

Step 4: 10.014 g of concentrate was taken and made up to 24.879 g. The solution was filtered through a 0.2μ filter.

TABLE 9

| Condition | Specification | Initial | 2° C. to 8° C. (3M) |
|---|---|---|---|
| Assay | 90%-110% | 100.5 | 99.8 |
| Impurity-E | NMT 1.0% | 0.04 | 0.05 |
| Impurity-L | NMT 1.0% | 0.09 | 0.17 |
| Impurity-J | NMT 1.0% | 0.01 | 0.09 |
| Impurity-F | NMT 1.0% | 0.11 | 0.39 |
| Impurity-I | NMT 1.0% | ND | ND |
| Impurity-U | NMT 1.0% | ND | ND |
| Highest Unknown | NMT 0.5% | 0.01 | 0.01 |
| Total impurities | NMT 3.0% | 0.39 | 0.91 |

### Example 10: Preparation of Ready to Use Aqueous Solution of Bortezomib (Form 2) with Only Light Control and No DO Control at 40° C.

Approximately 5.00 g of mannitol was weighed and added to 180.0 g of water with continuous stirring. To the solution, 1.8 g of sodium chloride was added and stirred continuously to obtain a clear solution. The solution was heated to a temperature of 40° C. To the heated solution, bortezomib (Form 2) API 0.50 g was added under continuous stirring to form a clear solution. The solution was cooled down to room temperature and final volume of the solution was made up to 200 g. All the operations were performed under sodium vapor lamp. The solution was filtered through a 0.2μ filter. 1.4 mL of solution was filled into each vial, stoppered with a rubber stopper and sealed with flip off seals.

TABLE 10

| Condition | Specification | Initial |
|---|---|---|
| Assay | 90%-110% | 100.4 |
| Impurity-E | NMT 1.0% | 0.07 |
| Impurity-L | NMT 1.0% | 0.04 |
| Impurity-J | NMT 1.0% | 0.04 |
| Impurity-F | NMT 1.0% | 0.05 |
| Impurity-I | NMT 1.0% | 0.01 |
| Impurity-U | NMT 1.0% | 0.01 |
| Highest Unknown | NMT 0.5% | 0.01 |
| Total impurities | NMT 3.0% | 0.25 |

### Example 11: Preparation of Ready to Use Aqueous Solution of Bortezomib (Form 2) with Only DO Control and No Light Control at 40° C.

Approximately 5.00 g of mannitol was weighed and added to 180.0 g of water with continuous stirring. To the solution, 1.8 g of sodium chloride was added and stirred continuously to obtain a clear solution. The solution was purged with nitrogen to maintain the dissolved oxygen below 2 ppm. The solution was heated to a temperature of 40° C. To the heated solution, bortezomib (Form 2) API 0.50 g was added under continuous stirring to form a clear solution. The solution was cooled down to room temperature and final volume of the solution was made up to 200 g. All the operations were performed under normal light. The solution was filtered through a 0.2μ filter. 1.4 mL of solution was filled into each vial, stoppered with a rubber stopper and sealed with flip off seals.

TABLE 11

| Condition | Specification | Initial |
|---|---|---|
| Assay | 90%-110% | 100.5 |
| Impurity-E | NMT 1.0% | 0.07 |
| Impurity-L | NMT 1.0% | 0.02 |
| Impurity-J | NMT 1.0% | 0.04 |
| Impurity-F | NMT 1.0% | 0.04 |
| Impurity-I | NMT 1.0% | 0.03 |
| Impurity-U | NMT 1.0% | 0.01 |
| Highest Unknown | NMT 0.5% | 0.01 |
| Total impurities | NMT 3.0% | 0.24 |

### Example 12: Preparation of Ready to Use Aqueous Solution of Bortezomib (Form 2) with Both DO Control and Light Control at 40° C.

Approximately 5.00 g of mannitol was weighed and added to 180.0 g of water with continuous stirring. To the solution, 1.8 g of sodium chloride was added and stirred continuously to obtain a clear solution. The solution was purged with nitrogen to maintain the dissolved oxygen below 2 ppm. The solution was heated to a temperature of 40° C. To the heated solution, bortezomib (Form 2) API 0.50 g was added under continuous stirring to form a clear solution. The solution was cooled down to room temperature and final volume of the solution was made up to 200 g. All the operations were performed under sodium vapor lamp. The solution was filtered through a 0.2μ filter. 1.4 mL of solution was filled into each vial, stoppered with a rubber stopper and sealed with flip off seals.

TABLE 12

| Condition | Specification | Initial |
|---|---|---|
| Assay | 90%-110% | 101.9 |
| Impurity-E | NMT 1.0% | 0.07 |
| Impurity-L | NMT 1.0% | 0.04 |
| Impurity-J | NMT 1.0% | 0.04 |
| Impurity-F | NMT 1.0% | 0.06 |
| Impurity-I | NMT 1.0% | 0.03 |
| Impurity-U | NMT 1.0% | ND |
| Highest Unknown | NMT 0.5% | 0.01 |
| Total impurities | NMT 3.0% | 0.28 |

### Example 13: Dilution Data at 1 mg/mL

For intravenous infusion, the bortezomib injection has to be administered at a concentration of 1 mg/mL, for which an aqueous solution of bortezomib 2.5 mg/mL was diluted to 1 mg/mL with 0.9% sodium chloride injection.

TABLE 13

| | | BOF002-17 | VELCADE |
|---|---|---|---|
| Condition | Specification | Initial | (lot#12161999) |
| Assay | 90%-110% | 102.4 | 100 |
| Impurity-E | NMT 1.0% | 0.03 | 0.18 |
| Impurity-L | NMT 1.0% | 0.33 | 0.05 |
| Impurity-J | NMT 1.0% | 0.08 | 0.05 |
| Impurity-F | NMT 1.0% | 0.13 | 0.18 |
| Impurity-I | NMT 1.0% | ND | ND |
| Impurity-U | NMT 1.0% | ND | ND |
| Highest Unknown | NMT 0.5% | 0.01 | 0.01 |
| Total impurities | NMT 3.0% | 0.62 | 0.54 |

### Example 14: Comparison of Ester Content for 2.5 mg/mL and 1 mg/mL with RLD

### VELCADE Sample Preparation:

Reconstitute by adding 1.4 mL 0.9% NaCl and shake well. Take 90% of the reconstituted solution and add 10% D20 solution and analyse the proton NMR by using 400 mHZ.

### IH Sample Preparation:

Take 90% product solution and add 10% D20 solution and analyse the proton NMR by using 400 mHZ.

TABLE 14

| Batch. No | 2.5 mg/mL |
|---|---|
| Inhouse sample | 62.1 |
| US RLD- VELCADE | 67.7 |

### Example 15: Preparation of Ready-to-Use Aqueous Bortezomib Composition Under Controlled Process Conditions

A liquid bortezomib composition was prepared in an aqueous medium under controlled process conditions. Approximately 2.5 g of mannitol was added to 90.0 g of water for injection (WFI) under continuous stirring to obtain a clear solution. To the solution, 0.9 g of sodium chloride was added and mixed until completely dissolved.

The solution was subjected to inert gas purging using nitrogen to reduce dissolved oxygen levels. Dissolved oxygen (DO) was monitored and maintained below 2 ppm prior to addition of the active ingredient.

Bortezomib (Form 2) API, 0.25 g, was added to the solution under continuous stirring at a controlled temperature not exceeding 30° C.

The composition was subjected to mechanical mixing using high shear homogenization. In certain embodiments, homogenization was initially performed at a shear rate corresponding to about 15 Hz to about 25 Hz to facilitate dispersion of the bortezomib particles, followed by homogenization at a shear rate corresponding to about 30 Hz to about 40 Hz until a visually clear solution was obtained.

The solution was filtered through a 0.2 μm filter and filled into glass vials under a nitrogen atmosphere such that headspace oxygen (HSO) was maintained below 1% by volume. The vials were stoppered and sealed. The resulting liquid bortezomib composition was substantially free of organic solvents. The composition was stored under refrigerated conditions (2° C. to 8° C.) and was observed for assay and impurity levels.

TABLE 15

| Initial Characteristics | | |
|---|---|---|
| Parameter | Specification | Initial |
| Assay | 90-110% | 101.3 |
| Impurity E | NMT 1.0% | 0.03 |
| Impurity L | NMT 1.0% | 0.12 |
| Impurity J | NMT 1.0% | 0.05 |
| Impurity F | NMT 1.0% | 0.06 |
| Impurity I | NMT 1.0% | ND |
| Impurity U | NMT 1.0% | ND |
| Highest Unknown | NMT 0.5% | 0.01 |
| Total Impurities | NMT 3.0% | 0.27 |

The impurity profile, including Impurity E, Impurity L, Impurity J, Impurity F, Impurity I, and Impurity U, remained within acceptable limits during storage, indicating improved stability of the composition. Thus, the formulation was free of solvent and exhibited low initial levels of degradation impurities.

### Example 16: Stability of Liquid Bortezomib Composition Prepared Under Controlled Process Conditions

A liquid bortezomib composition prepared according to the process described in Example 16 was evaluated for stability under refrigerated storage conditions (2° C. to 8° C.).

The composition was analyzed at predetermined intervals to assess assay and impurity levels.

TABLE 16
Stability Data (2° C. to 8° C.)

| Parameter | Limits % | Initial | 6 Months | 12 Months | 24 Months |
|---|---|---|---|---|---|
| Assay (%) | 90-110% | 101.3 | 100.8 | 100.2 | 99.6 |
| Impurity E (%) | NMT 1.0% | 0.03 | 0.05 | 0.07 | 0.09 |
| Impurity L (%) | NMT 1.0% | 0.12 | 0.18 | 0.25 | 0.32 |
| Impurity J (%) | NMT 1.0% | 0.05 | 0.08 | 0.11 | 0.14 |
| Impurity F (%) | NMT 1.0% | 0.06 | 0.09 | 0.12 | 0.15 |
| Highest Unknown (%) | NMT 1.0% | 0.01 | 0.01 | 0.02 | 0.02 |
| Total Impurities (%) | NMT 3.0% | 0.27 | 0.41 | 0.58 | 0.72 |

The assay of bortezomib remained within acceptable limits throughout the storage period.

Degradation impurities, including Impurity E, Impurity L, Impurity J, Impurity F, were monitored and found to remain substantially lower than the specified limits over the storage period. Total impurities were maintained well within acceptable limits during storage, demonstrating that the composition exhibited controlled impurity growth under refrigerated conditions.

The stability profile observed is consistent with the controlled process conditions employed during preparation, including control of temperature, high shear homogenization, dissolved oxygen, and headspace oxygen. The composition exhibited stability of up to about 24 months under refrigerated conditions, while maintaining acceptable assay and impurity levels. The results demonstrate that simultaneous control of mechanical dissolution conditions, dissolved oxygen, headspace oxygen, and reduced thermal exposure suppresses impurity growth during long-term refrigerated storage.

### Example 17: Evaluation of Dose Utilization and Residual Drug in Liquid Bortezomib Compositions

The effect of vial strength and fill volume on drug utilization was evaluated for liquid bortezomib compositions administered according to body surface area (BSA)-based dosing regimens.

Bortezomib is typically administered at a dose of 1.3 mg/m². Based on this dosing regimen, the required dose varies depending on patient BSA. Fixed vial strengths may therefore result in residual unused drug after withdrawal of the required dose.

A liquid bortezomib composition according to the present invention, comprising bortezomib at a concentration of about 2.5 mg/mL in a fill volume corresponding to a total drug content of about 3 mg, was evaluated across a BSA range of about 1.4 m² to about 2.2 m².

The amount of drug required per patient was calculated based on the dosing regimen, and the residual drug remaining in the vial after withdrawal of the required dose was determined.

The residual drug associated with the 3 mg presentation was compared with that associated with higher-strength vial presentations.

TABLE 17
Residual Drug Comparison

| | Dose | 3.5 mg Vial | 3 mg Vial | |
|---|---|---|---|---|
| BSA | Required | Residual | Residual | % |
| (m²) | (mg) | (mg) | (mg) | Reduction |
| 1.4 | 1.82 | 1.68 | 1.18 | 29.8% |
| 1.6 | 2.08 | 1.42 | 0.92 | 35.2% |
| 1.8 | 2.34 | 1.16 | 0.66 | 43.1% |
| 2.0 | 2.60 | 0.90 | 0.40 | 55.6% |
| 2.2 | 2.86 | 0.64 | 0.14 | 78.1% |

It was observed that the 3 mg presentation resulted in improved dose alignment across the evaluated BSA range, leading to a reduction in residual drug remaining after administration.

The residual drug remaining after administration was reduced by at least about 30% to about 78% compared to higher-strength presentations.

The results demonstrate that the 3 mg presentation provides improved dose alignment and reduced residual drug compared to conventional higher-strength presentations.

What is claimed is:

1. A liquid pharmaceutical composition comprising:
from about 1 mg/mL to about 5 mg/mL bortezomib;
from about 15 mg/mL to about 40 mg/mL mannitol;
from about 5 mg/mL to about 18 mg/mL sodium chloride; and
an aqueous carrier;
wherein the composition is substantially free of organic solvents;
wherein after storage for at least about 12 months at about 2° C. to about 8° C., the composition comprises:
not more than about 0.3 wt. % of Impurity E, Impurity J, or Impurity F, individually;
not more than about 0.5 wt. % of Impurity L; and
not more than about 1.0 wt. % of total impurities;
wherein the composition is prepared by a process comprising:
mechanical mixing or high shear homogenization performed at shear rates corresponding to about 15 Hz to about 40 Hz at a temperature not exceeding about 30° C.

2. The liquid pharmaceutical composition according to claim 1, wherein the composition comprises:
about 2.5 mg/mL of bortezomib;
about 25 mg/mL of mannitol; and
about 9 mg/mL of sodium chloride.

3. The liquid pharmaceutical composition according to claim 1, wherein after storage for at least about 24 months at about 2° C. to about 8° C., the composition comprises:
not more than about 0.3 wt. % of Impurity E, Impurity J, or Impurity F, individually;
not more than about 0.5 wt. % of Impurity L; and
not more than about 1.0 wt. % total impurities.

4. The liquid pharmaceutical composition according to claim 1, wherein the composition is provided at a concentration of about 2.5 mg/mL in a fill volume corresponding to a total drug content of about 3 mg bortezomib in a container suitable for subcutaneous or intravenous administration.

5. The liquid pharmaceutical composition according to claim 4, wherein the composition provides reduced residual drug following administration compared to a 3.5 mg bortezomib presentation across a body surface area range of about 1.4 m² to about 2.2 m².

6. The liquid pharmaceutical composition according to claim 5, wherein residual drug is reduced by at least about 10%.

7. The liquid pharmaceutical composition according to claim 6, wherein residual drug is reduced by at least about 20%.

8. The liquid pharmaceutical composition according to claim 7, wherein the reduction in residual drug is from about 30% to about 80%.

9. The liquid pharmaceutical composition according to claim 1, wherein the composition is suitable for subcutaneous or intravenous administration.

10. The liquid pharmaceutical composition according to claim 1, wherein dissolved oxygen is maintained at not more than about 5 ppm during preparation of the composition.

11. The liquid pharmaceutical composition according to claim 10, wherein dissolved oxygen is maintained at not more than about 2 ppm.

12. The liquid pharmaceutical composition according to claim 1, wherein headspace oxygen is maintained at not more than about 5%.

13. The liquid pharmaceutical composition according to claim 12, wherein headspace oxygen is maintained at not more than about 2%.

14. The liquid pharmaceutical composition according to claim 1, wherein the composition is filled under an inert gas atmosphere.

15. A liquid pharmaceutical composition comprising:
from about 1 mg/mL to about 5 mg/mL bortezomib;
from about 15 mg/mL to about 40 mg/mL mannitol;
from about 5 mg/mL to about 18 mg/mL sodium chloride; and
an aqueous carrier;
wherein the composition is substantially free of organic solvents; and
wherein the composition is prepared by a process comprising the following steps:
(a) preparing a solution comprising the mannitol, the aqueous carrier, and the sodium chloride;
(b) purging the solution from step (a) with an inert gas;
(c) adding the bortezomib to the solution of step (b) at a temperature not exceeding about 30° C.;
(d) mixing the product of step (c) at a shear rate of from about 15 Hz to about 40 Hz until a clear solution is obtained; and
(e) filling the product of step (d) in a vial under an inert gas atmosphere; and
wherein after storage for at least about 12 months at about 2° C. to about 8° C., the composition comprises:
not more than about 0.3 wt. % of Impurity E, Impurity J, or Impurity F, individually;
not more than about 0.5 wt. % of Impurity L; and
not more than about 1.0 wt. % totally impurities.

16. The liquid pharmaceutical composition according to claim 15, wherein the temperature does not exceed about 25° C. during preparation of the composition.
